(19) United States
(12) Patent Application Publication
(10) Pub. No.: US 2007/0033677 A1
(43) Pub. Date: Feb. 8, 2007

(54) Plant sHSP gene bidirectional promoter and uses thereof

(75) Inventors: Chu-Yung Lin, Taipei (TW); Jiahn-Chou Guan, Taipei (TW)

Correspondence Address: TROXELL LAW OFFFICE PLLC, 5205 LEESBURG PIKE, SUITE 1404, FALLS CHURCH, VA, 22041, US

(73) Assignee: National Taiwan University

(21) Appl. No.: 11/312,711

(22) Filed: Dec. 21, 2005

(30) Foreign Application Priority Data
Aug. 2, 2005 (TW) 94126214

(51) Int. Cl. A01H 5/00; C12N 15/82; C12N 5/04; A01H 1/00

(52) U.S. Cl. 800/288; 800/289; 435/468; 435/419; 800/320.2

(57) ABSTRACT

A plant sHSP gene bidirectional promoter and uses thereof. This invention relates to the use of a single bidirectional promoter from rice sHSP gene to regulate expression of two separate genes linked in opposite orientations. More particularly it relates to the use of said bidirectional promoter to regulate expression of two separate genes for applications which require production of two separate gene products in the same cell, including applications which require induction of said two separate gene products under heat shock, or chemical inducers.

### BACKGROUND OF THE INVENTION

(1) Field of the Invention

The present invention relates to plant sHSP (small heat shock protein) gene, especially for a plant sHSP gene promoter which could function as a bidirectional promoter.

(2) Description of the Prior Art

Heat shock (HS) response is a conserved physiological phenomenon invoked in all organisms by a sudden increase in temperature. HS response is characterized by elevated synthesis of a set of HSPs (Heat shock proteins) and repressed synthesis of most normal proteins and mRNAs. A common feature of the HS response is to develop acquired thermotolerance. Plants, like other organisms, have the ability to acquire thermotolerance rapidly. Many studies have documented that induction of HSPs is well correlated with the acquired thermotolerance in a time- and temperature-dependent manner. Based on this correlation, it has been hypothesized that accumulation of HSPs is an essential component of processes in preventing and recovering from heat damages (Key et al., 1981; Lin et al., 1984; Kimpel and Key, 1985).

HSPs comprise several evolutionarily conserved protein families including ClpB/HSP100, HSP90, HSP70/DnaK, HSP60/chaperonin, and small HSP (sHSP).

sHSPs are the most abundant and complex subset of HSPs in plants and their synthesis is induced by a rapid increase of temperature. They are encoded by members of a multi-gene family in eukaryotes and defined by possessing a conserved α-crystallin domain (ACD). They are divided into at least six classes based on amino acid sequence homology, immunological cross-reactivity, and subcellular localization. Class II, and III sHSPs are present in both cytosol and nucleus. Members of the other three classes are localized in the plastids, endomembranes, and mitochondria.

All major classes of HSPs are proposed to act as molecular chaperones, functioning through binding to substrate proteins that are in unstable, normative conformational states. By virtue of this property, the different HSPs comprise a multi-chaperone network to aid in a variety of cellular processes that involve assisted protein folding, including prevention of denatured proteins from aggregation or rescue of aggregated proteins. These activities explain their important roles in heat stress that leads to extensive protein denaturation. Plants synthesize all spectra of HSPs in response to heat stress, but the specific contributions of the different members of the HSP superfamily as to functional activities in the complex network of plant heat shock response are distinct.

Although sHSPs have been found only under stress conditions in vegetative tissues; however, there are a few examples of constitutive synthesis of sHSPs with tissue-specific distribution and cellular localization in vegetative tissues in the absence of stress. The function of sHSPs in the vegetative plant organs is not clear yet. They suggest that the expression of non-heat-induced sHSPs seems to be essential for accumulation of large amounts of storage proteins in perennial plant vegetative storage organs (Lubaretz and Nieden, 2002).

In addition to HS and developmental cues, many recent studies indicate that sHSPs are also regulated by a variety of environmental stresses other than heat stress in animals (Snoeckx et al., 2002) and plants (Waters et al., 1996; Sun et al., 2002). For understanding the regulation of HSP gene expression and cross-tolerance in higher plants, various chemical agents were widely used for studying the expression of plant HSP genes. Chemical inducers such as ethanol (Kuo et al., 2000), amino acid analogs (Lee et al., 1996), ozone (Banzet et al., 1998) and also heavy metals such as arsenite and cadmium (Lin et al., 1984; Edelman et al., 1988; Tseng et al., 1993) were used for induction of one subset of sHSP genes.

Several sHSP-encoding genes are also induced by cold stress, photoperiod, UV radiation, and γ-irradiation (Waters et al., 1996; Sun et al., 2002). Recent microarray studies in Arabidopsis also revealed that a subset of sHSP genes was induced by various stresses such as salt, drought, chilling, oxidative stress, and wounding (Desikan et al., 2001; Cheong et al., 2002; Becker et al., 2003).

The expression of the heat shock genes is mainly attributed to activation of the heat shock factors (Hsf) under heat stress. Hsfs as trimers recognize the highly conserved HSE, which has been defined as adjacent and inverse repeats of the motif 5′-nGAAn-3′, such as 5′-nGAAnnTTCnnGAAn-3′ (Schoffl et al., 1998). In addition to heat stress, ethanol (Kuo et al., 2000), amino acid analogs (Lee et al., 1996), chilling (Sabehat et al., 1998) and heavy metals such as As and Cd (Lin et al., 1984; Edelman et al., 1988; Tseng et al., 1993) also induce expression of one subset of sHSP genes. Recent microarray studies in Arabidopsis revealed that a subset of sHSP genes was induced by various stresses such as salt, drought, chilling, oxidative stress, and wounding (Desikan et al., 2001; Cheong et al., 2002). Moreover, members of the sHSP gene families are also developmentally regulated in seeds, storage organs, and vegetative tissues in plants (Wehmeyer and Vierling, 2000; Lubaretz and Nieden, 2002; Jofre et al., 2003). The chaperone function of sHSP is usually emphasized under heat stress condition; however, the versatile expression patterns strongly suggest that sHSP may be important for other stresses and developmental conditions. Although it is known that the above described stresses elicit sHSP expression, the molecular mechanisms underlying the induction and the relationship between heat stress and other stresses remain unclear.

Rice plant is sensitive to heat stress at all stages of development (Maestri et al., 2002). Because of the distinct abundance and complexity of sHSP-CI in rice, much research has concentrated on the identification of sHSP-CI genes in our laboratory Tseng et al., 1993; Tzeng et al., 1993; Lee et al., 1995; Chang et al., 2001; Guan et al., 2003). In reports, we identified and characterized nine members of the rice sHSP-CI gene family on chromosome 1 and 3 and examined during seed maturation and the effects of various stresses including HS, amino acid analogs, As, Cd, and ethanol on expression profiles of these genes in etiolated seedlings. Our results indicate that different mechanisms may be involved in the selective induction of sHSP-CIs by heat stress and chemical agents. The research group of the inventors report the characterization and the expression profile of 9 members of the sHSP-CI gene family in rice (Oryza sativa Tainung No.67), of which Oshsp16.9A, Oshsp16.9B, Oshsp16.9C, Oshsp16.9D and Oshsp17.9B are clustered on chromosome 1, and Oshsp17.3, Oshsp17.7, Oshsp17.9A and Oshsp18.0 are clustered on chromosome 3. The rice sHSP-CI genes share high homology in the coding regions (>60%) and low homology in the 3′-UTRs (<40%).

### SUMMARY OF THE INVENTION

The object of the present invention is to provide a plant sHSP gene, especially a plant sHSP gene consisting of SEQ ID NO:5 which could function as a bidirectional promoter.

Another object of the present invention is to provide a bidirectional promoter consisting of SEQ ID NO:5 to regulate expression of two separate genes for applications which require production of two separate gene products in the same cell.

According, the present invention relates to a plant sHSP gene bidirectional promoter and uses thereof. The use of a single bidirectional promoter from rice sHSP gene to regulate expression of two separate genes linked in opposite orientations. More particularly it relates to the use of said bidirectional promoter to regulate expression of two separate genes for applications which require production of two separate gene products in the same cell, including applications which require induction of said two separate gene products under heat shock, or chemical inducers.

### BRIEF DESCRIPTION OF THE DRAWINGS

The present invention will now be specified with reference to its preferred embodiment illustrated in the drawings, in which

FIG. 1 shows the total 567 bps nucleotide sequences of SEQ ID NO:5.

FIG. 2A shows the photographs of the leaves which comprises Oshsp 18.0 promoter activity.

FIG. 2B shows the photographs of the leaves which comprises Oshsp 17.3 promoter activity.

FIG. 3A shows the result of GUS activity assay.

FIG. 3B shows the photograph that the bombarded coleoptiles are stained for GUS activity.

### DESCRIPTION OF THE PREFERRED EMBODIMENT

The invention discloses a plant sHSP gene bidirectional promoter and uses thereof. This invention relates to the use of a single bidirectional promoter from rice sHSP gene to regulate expression of two separate genes linked in opposite orientations. More particularly, it relates to the use of said bidirectional promoter to regulate expression of two separate genes for applications which require production of two separate gene products in the same cell, including applications which require induction of said two separate gene products under heat shock, or chemical inducers.

The term “bidirectional promoter” as used herein is defined as a promoter which directs transcription of specific nucleotide sequences in opposite orientations. That is, it directs transcription of a specific nucleotide sequence which lies 5′ to 3′ in the same 5′ to 3′ direction as said promoter and it directs transcription of another specific nucleotide sequence which lies 5′ to 3′ in a direction opposite from the 5′ to 3′ direction of said promoter. The nucleotide sequences are in fixed positions relative to the promoter sequence with the 5′ ends of said nucleotide sequences always positioned most proximal to the promoter. However, the orientation of said promoter can be reversed relative to its position between said diverging nucleotide sequences and still allow promoter activity.

For detailed description of the invention, several embodiments of the invention are described as followed. While the present invention has been particularly shown and described with reference to a preferred embodiment, it will be understood by those skilled in the art that various changes in form and detail may be without departing from the spirit and scope of the present invention.

What follows introduces the brief experiment steps about how to sieve out the bidirectional promoter sequence from rice (Oryza sativa L. cv. Tainung No. 67) and some experiments to prove the function of the bidirectional promoter sequence disclosed herein.

Plant Materials

Rice (Oryza sativa L. cv. Tainung No. 67) seedlings were germinated in rolls of moist paper towels at 28° C. in a dark growth chamber. Tainung No.67 belongs to the japonica subspecies and is widely grown in paddy fields in Taiwan. Three-day-old rice seedlings without endosperms were incubated in shaking buffer (1% (w/v) sucrose and 5 mM potassium phosphate buffer pH 6.0) in shaking baths at various temperature regimes. For seed development, rice plants were grown in a 28° C. growth chamber with a 16-h day length. For chemical stress treatments, seedlings were incubated at 28° C. in shaking buffer with added chemicals as indicated. Samples were harvested and flash-frozen in liquid nitrogen and stored at 80° C. for subsequent RNA or protein extraction.

RNA and Genomic DNA Isolation

Samples were ground in liquid nitrogen using a mortar and pestle. Total RNA was extracted using TRIZOL reagent (Invitrogen, Rockville, Md., USA) according to the manufacturer's protocol. Genomic DNA was extracted using DNAZOL reagent (Life technologies/Gibco-BRL, Cleveland) according to the manufacturer's protocol.

Construction and Screening of Genomic Libraries for sHSP-CI Genes

Total rice genomic DNA was digested with restriction enzyme, EcoR I, separated on agarose gel and the DNA fragments sizes between 1 and 2 kb were eluted from the agarose gel. The size-selected genomic library was established in Lambda ZAP Express EcoR I/CIAP-treated vector, using the ZAP Express Gigapack II Gold cloning kit (Stratagene, La Jolla, Calif., USA) according to the manufacturer's protocol. The library was screened by hybridization with ³²P-labeled cDNA pTS1 probes (>10⁷ cpm). Filters were prehybridized in 50% formamide, 5×SSC, 0.1% SDS, 20 mM sodium phosphate, pH 6.5, 0.1% Ficoll, 0.1% polyvinylpyrolidone, 1% glycine, 250 μg/ml denatured salmon sperm DNA at 42° C. for at least 2 hours. Hybridization was performed at 42° C. for overnight in the prehybridized solution with ³²P-labeled probes (>10⁷ cpm specific activity, cpm/ug DNA). The probes were labeled with (α-³²P)-dCTP (1000 Ci/mmol, Amersham, Buckinghamshire, UK) using Prime-a-Gene Labeling System (Promega, Madison, Wis., USA). Then, the filters were washed three times in 3×SSC, 0.1% SDS at room temperature for 10 minutes with two washes in 0.1×SSC, 0.1% SDS at 56° C. for 30 minutes each. The inserts from positive clones were in vivo excised from the ZAP Express vector and maintained in the pBK-CMV phagemid vector (Stratagene, La Jolla, Calif., USA) according to the manufacturer's protocol. The DNA sequence was determined by using the Sequenase version 2.0 DNA sequencing kit (USB, Cleveland, Ohio, USA) according to the manufacturer's protocol. The University of Wisconsin Genetics Computer Group (UWGCG) program suite was used to perform the sequence analysis through the NHR1 of Taiwan at Nankang, Taipei.

PCR

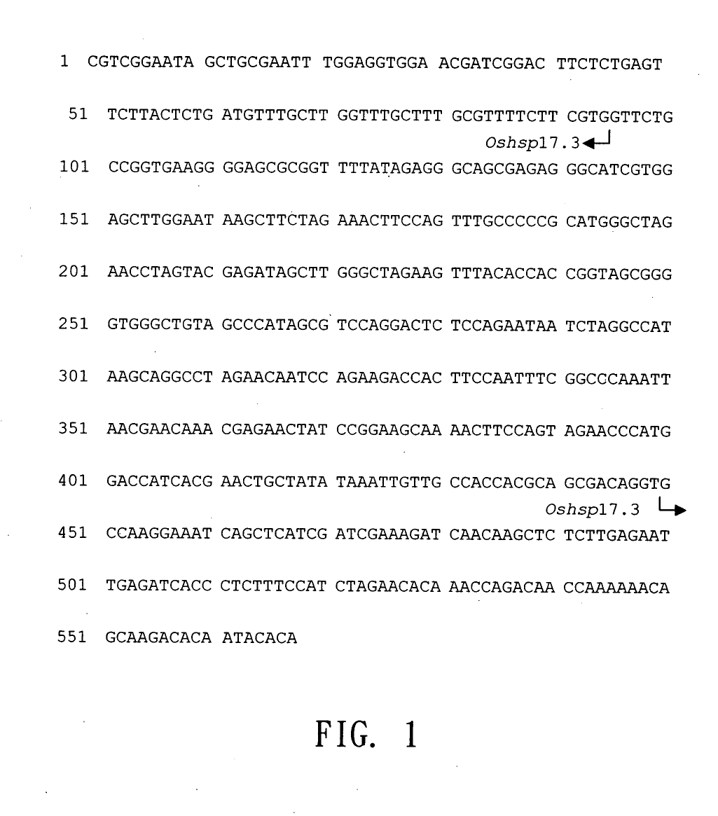

Rice genomic DNA digested with Eco RI was used as templates for PCR with gene specific primers. Herein the specific primers comprises SEQ ID NO:1 and SEQ ID NO:2, which consisting the coding region of 5′-UTR (untranslated region) of Oshsp17.3 and Oshsp18.0, the primer consisting of SEQ ID NO:1 is a forward primer and the primer consisting of SEQ ID NO:2 is a reverse primer. The product from above PCR procedures is a 567 bps nucleotide sequence consisting of SEQ ID NO:5, which comprises a 93 bps 5′-UTR coding region of Oshsp17.3, a 119 bps 5′-UTR coding region of Oshsp18.0, and a 355 bps bidirectional promoter region. The specific forwards primer and the reverse primer are consisting of SEQ ID NO:3 and SEQ ID NO:4 respectively. Please refer to FIG. 1, which shows the total 567 bps nucleotide sequences of SEQ ID NO:5.

Semi-Quantitative RT-PCR

Firstly, total RNA (1 μg) was treated with one unit of DNase I (Promega, Madison, Wis., USA) for 15 min at room temperature prior to RT-PCR to remove residual DNA contamination. The RT-PCR analyses were conducted using Superscript one-step RT-PCR kit (Invitrogen, Rockville, Md., USA) according to the manufacturer's protocol. Primers were designed to yield PCR products with lengths between 150 to 240 bp. Sixteen ng of total RNA were reverse transcribed into cDNA using random primer, d(N)₆, and then amplified with gene (Oshsp17.3 and Oshsp18.0) specific primers (10 pmole for each primer) in the same tube. For each RT-PCR reaction, a pair of plant 18S rRNA internal standard primers (Ambion, Austin, Tex., USA) was conducted as an internal PCR control.

PCR reactions for all genes were subjected to 25 cycles of 95° C. (30 s), 54° C. (45s), and 72° C. (45 s) with GeneAmp PCR System 2400 (Perkin-Elmer Applied Biosystems, Foster City, Calif., USA). For all treatments, three replicates of RT-PCR were conducted with three batches of total RNA samples isolated independently.

DNA from 20 μl of each PCR reaction was fractionated by electrophoresis through agarose gel with 0.5% (w/v) ethidium bromide in 1.5% Tris-borate EDTA buffer. The gels were digitally photographed with a FloGel-1 fluorescent gel digital imaging system (TOPBIO, Taipei, Taiwan). Scion Image for Windows (Scion, http://www.scioncorp.com) software was used to quantify the intensity of the ethidium bromide stained DNA bands from the negative images of the gels.

Vector Construction

Herein we choose three kind of vectors to be cloned the function bi-directional promoter sequence; pCAMBCIA1381Z vectors, pCAMBIA1391Z vectors, and pGN100 vectors bearing the β-glucuronidase (gus) gene fused with nos termination sequence (Jinn et al., 1999). All the promoter regions contain the regulatory sequence and 5′-UTR. All of the constructs were verified by restriction enzyme digestions and sequencing.

Transgenic Plants

The vectors pCAMBCIA1381 Z-promoter::GUS and pCAMBIA1391Z-promoter::GUS are transfer into Arabidopsis via floral dipping method. Wherein the transgenic seeds are sieved via MS plate comprising kanamycin 50 μg/ml, and leaf PCR is used to testify successfully transformed.

Kinds of environmental stresses are used to test the induction of bidirectional promoter expression, in series of test experiments, the concentration of stresses such as Cu, As, Cd, amino acid analogs, NaCl, ethanol, and H₂O₂ are 500 μM [symbol] 250 μM [symbol] 1 mM [symbol] 5 mM 300 mM [symbol] 5% [symbol] 0.03% respectively. Furthermore, the temperature of heat stress is in a range of 32° C. to 48° C. herein.

GUS Activity Assay

For testing the function of the bidirectional promoter sequence, kinds of environmental stresses are used to test the transgenic Arabidopsis plants. Herein a heat stress is used to analysis GUS activity.

The Arabidopsis leaves are incubated at 41° C. in a phosphorous solution for 2 hours. Then treatment with fixing solution (0.1 M NaPO4 pH7.0 [symbol] 0.1% formaldehyde 0.1% Triton X-100 and 0.1% β-mercaptoethanol). Wherein the staining solution is composed of 0.1 M NaPO₄ pH7.0 [symbol] 10 mM EDTA [symbol] 5 mM potassium ferricyanide [symbol] 5 mM potassium ferrocyanide [symbol] 1 mM X-glucuronide and 0.1% Triton X-100. After dyeing, the finally treatment is using ethanol to fade segments away.

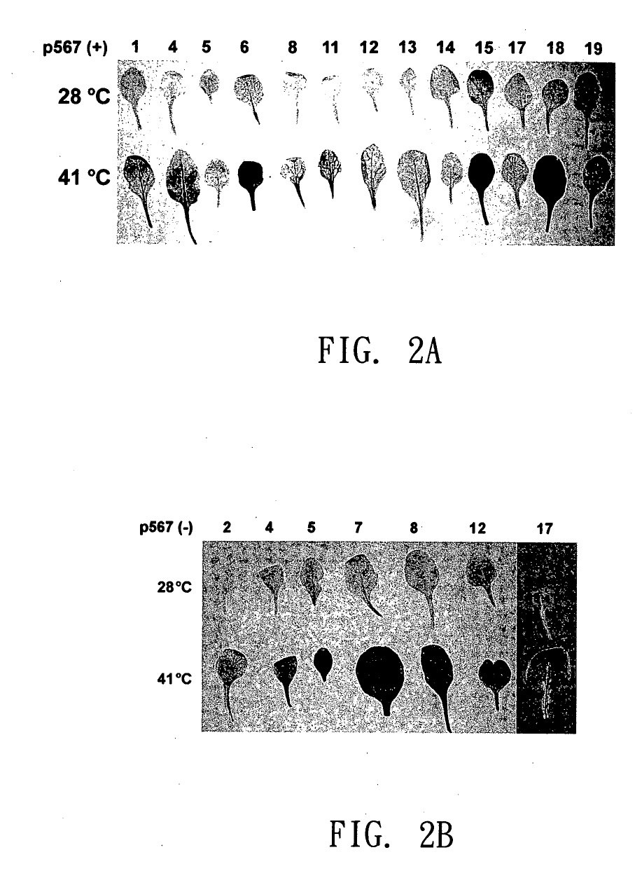

Please refer to FIG. 2A and FIG. 2B, which are photographs of the experimental Arabidopsis leaves. FIG. 2A shows the photographs of the leaves which comprises Oshsp18.0 promoter activity, and FIG. 2B shows the photographs of the leaves which comprises Oshsp 17.3 promoter activity. Arabidopsis leaves incubated at 28° C. are control groups in both of the two photographs. From the photographs we can see both the two promoters could induce the transcription of the GUS reporter gene, it means that the nucleotide sequence comprising SEQ ID NO:5 really could play as a role of bidirectional promoter.

Transient Expression System

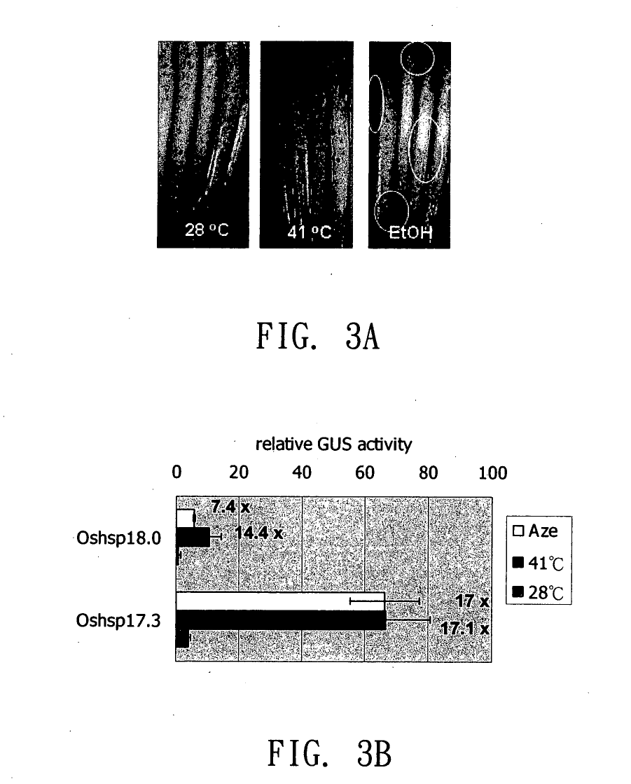

The coleoptile of a etiolated rice seedling was cut from embryonic root and positioned on the middle of a 10-cm Petri dish containing MS salts supplemented with 0.6% (w/v) agarose and 3% (w/v) sucrose. The mixture (in a 1:1 molar ratio) of a test DNA construct and a maize ubiquitin-luciferase internal control construct were coated onto the gold particles as follows: under continuous vortexing, the following were added in order to each 10-μL aliquot of 3 mg gold particles (Shen et al., 1996): 5 μL of DNA (1 μg DNA), 10 μL of 2.5 M CaCl₂, and 4 μL of 0.1 M spermidine (free-based, tissue-culture grade). Gold microcarriers (1.6-μm particle size, 30 mg) in a microcentrifuge tube were vortexed with 1 nL 70% (v/v) ethanol for 3˜5 minutes and kept till precipitation for about 15 minutes. Then, after centrifuging for 5 seconds, the supernatant was discarded and washed three times with 1 ml of sterile de-ionized water. The gold particles were resuspended in 500 μL 50% (w/v) glycerol, and then dispensed in 50-μL aliquots (3 mg/50 μL) kept in −20° C. The gold particles coated with DNA were pelleted in an bench-top Eppendorf centrifuge at maximum speed for 5 seconds, discarded the supernatant, washed once with 70% (v/v) ethanol following by absolute ethanol, and resuspended in 20 μL absolute ethanol. The 20-μL DNA-coated gold particles was pipetted and sprayed onto the center of macrocarriers and dried in air. A helium biolistic particle-delivery system (model PDS-1000, Bio-Rad, Hercules, Calif., USA) was used for particle bombardment. The bombardment parameters optimized included He pressure, gap distance (the distance from the power source to the macroprojectile), and the target distance (the distance from microprojectile launch site to the sample target). All bombardments were performed at 1,350 psi under a vacuum of 26 mm Hg, with a distance of 6 cm between the targets and the barrel of the particle gun. Following the bombardments, the Petri dishes were incubated at 28° C. in the dark for at least 6 h and then subjected to experimental treatments indicated. After incubation under heat shock or 5 mM Aze, separately, for 2 and 4 h, the bombarded coleoptiles were homogenized in 600 μl grinding buffer (Shen et al., 1996). After centrifugation at 12,000×g at 4° C. for 15 min, 50 μl of the supernatant was assayed for luciferase activity by Bright-Glo™ luciferase assay system (Promega, Madison, Wis., USA) according to the technical manual. The luminescence was detected by an OPTOCOMP I luminometer (MGM Instruments, CT, USA). For the GUS activity assay, 50 μl of the supernatant was diluted into 200 μl of GUS assay buffer (Shen et al. 1996) and incubated at 37° C. for 20 h. One hundred microliters of the reaction mixture was then diluted into 900 μl of 0.2 M Na₂CO₃ (pH 11.2). After aliquot every 300 μl into three separate wells of a 96-well plate, the resulting fluorescence was measured in a Fluoroskan Ascent FL fluorometer (Labsystems, Helsinki, Finland). Normalized GUS activity was calculated by dividing GUS activity by luciferase activity of each respective sample. To test whether the selective induction of sHSP genes by Aze treatment observed in vivo was evoked by the differences related to promoter activity, we prepared two promoter::GUS constructs for transient expression assays by bombarded to rice coleoptiles. The promoter::GUS constructs contained the 567-bp promoter region of Oshsp17.3 and Oshsp18.0 on chromosome 3. Please refer to FIG. 3A, which shows the result of GUS activity assay. The GUS activities of all samples were normalized against those of a luciferase internal control. Bombarded coleoptiles were incubated for at least 6 hours at 28° C., and then the samples were transferred to shaking buffer for 2 hours HS treatment or 4 hours Aze treatment. As FIG. 3A shows, the Oshsp17.3 or Oshsp18.0 promoter was induced over 14-fold by HS and at least 7-fold by Aze treatment. The results of transient expression assays supported the in vivo selective expression of sHSP-CI genes by Aze treatment indicating that the promoter activity is involved in differential transcription.

Please refer to FIG. 3B, which shows the photograph that the bombarded coleoptiles are stained for GUS activity assay. From FIG. 3B we can see the coleoptiles via heat stress (41° C.) and 5% ethanol treatment express much more amount of reporter gene, wherein the coleoptiles under 28° C. is a control group. From above, we can see that the nucleotide sequences of SEQ ID NO:5 do play the role of bidirectional promoter. The bidirectional promoter could regulate expression of two separate genes linked in opposite orientations. Moreover, the character that the nucleotide sequences of SEQ ID NO:5 may be effected by various environmental stresses such as HS, Aze, As, Cd, and ethanol on expression profiles indicates that different applications may be working. For example, for detecting the pollution of heavy metal in soils, or the differential change during the growth of plants. Another kinds of application could be workable by operatively linking different heterogeneous genes.

We claim:

1. A nucleotide sequence comprises SEQ ID NO:5.

2. A nucleotide sequence comprising SEQ ID NO:5, wherein the nucleotide sequence functions as a bidirectional promoter.

3. A recombinant vector comprising the nucleotide sequence of claim 2.

4. The recombinant vector of claim 3 further comprising two DNA fragments coding for heterologous polypeptides, wherein the two DNA fragments are separately linked to each end of the nucleotide sequence.

5. The recombinant vector of claim 4, wherein the nucleotide sequence drives transcription of the two DNA fragments.

6. The recombinant vector of claim 4, wherein the two DNA fragments coding for the same or different products.

7. The recombinant vector of claim 4, wherein the nucleotide sequence drives transcription of the two DNA fragments simultaneously.

8. A transformed plant cell comprising the nucleotide sequence of claim 1.

9. The transformed plant cell of claim 8 is an angiosperm cell.

10. The transformed plant cell of claim 8 is a rice cell.

11. The transformed plant cell of claim 8, wherein the nucleotide sequence regulates the transcription of the two DNA fragments within the transformed plant cell.

12. The transformed plant cell of claim 11, wherein a heat stress could induce the nucleotide sequence to drive the transcription of the two DNA fragments.

13. The transformed plant of claim 11, wherein the nucleotide sequence could induce the transcription of the DNA fragments by chemical stresses.

14. The transformed plant of claim 13, wherein the chemical stresses selected from the group of Cu, As, Cd, ethanol, NaCl, amino acid analogs, and H₂O₂.

15. The transformed plant of claim 14, wherein the amino acid analog is L-azetidine-2-carboxylic acid.

16. The transformed plant of claim 14, wherein the amino acid analog is canavanine.

17. The transformed plant of claim 8, wherein the recombinant vector further comprises a reporter gene.

18. The transformed plant of claim 17, wherein the reporter gene is β-glucuronidase gene.

19. A method of producing two heterologous polypeptides within an angiosperm plant cell, comprising:
(a) constructing a recombinant vector which comprises a Nucleotide sequence SEQ ID NO:5 and two DNA fragments coding for heterologous polypeptides, wherein the Nucleotide sequence works as a bidirectional promoter;
(b) Transformation of the recombinant vector into the angiosperm plant cell
(c) culture of the angiosperm plant cell; and
(d) application of an environmental stress to the cultured angiosperm plant cell to induce the bidirectional promoter driving the transcription of the two DNA fragments.

20. The method of claim 19, wherein the transformation system is Agrobacterium-mediated transformation, PEG-mediated transformation, particle bombardment-mediated transformation, electroporation-mediated transformation, sonication-mediated transformation, or micro-injection.

21. The method of claim 20, wherein the angiosperm plant cell could be suspension cells of rice, barley, or wheat.

22. The method of claim 19, wherein the environmental stress comprises heat stress and chemical stress.

23. The method of claim 22, wherein the temperature of heat stress is in a range of 32° C. to 48° C.


SEQUENCE LISTING (6 sequences)

SEQ ID NO: 1 - DNA, 25 bp, Artificial Sequence
  Description of Artificial Sequence Synthetic primer
cgtcggaatagctgcgaatttggag

SEQ ID NO: 2 - DNA, 25 bp, Artificial Sequence
  Description of Artificial Sequence Synthetic primer
tgtgtattgtgtcttgtgttttttg

SEQ ID NO: 3 - DNA, 21 bp, Artificial Sequence
  Description of Artificial Sequence Synthetic primer
ggttctgccggtgaaggggag

SEQ ID NO: 4 - DNA, 22 bp, Artificial Sequence
  Description of Artificial Sequence Synthetic primer
cctgtcgctgcgtggtggcaac

SEQ ID NO: 5 - DNA, 567 bp, Artificial Sequence
  Description of Artificial Sequence Synthetic DNA construct
cgtcggaatagctgcgaatttggaggtggaacgatcggacttctctgagttcttactctgatgtttgcttggtttgctttgcgttttcttcgtggttctgccggtgaaggggagcgcggttttatagagggcagcgagagggcatcgtggagcttggaataagcttctagaaacttccagtttgcccccgcatgggctagaacctagtacgagatagcttgggctagaagtttacaccaccggtagcggggtgggctgtagcccatagcgtccaggactctccagaataatctaggccataagcaggcctagaacaatccagaagaccacttccaatttcggcccaaattaacgaacaaacgagaactatccggaagcaaaacttccagtagaacccatggaccatcacgaactgctatataaattgttgccaccacgcagcgacaggtgccaaggaaatcagctcatcgatcgaaagatcaacaagctctcttgagaattgagatcaccctctttccatctagaacacaaaccagacaaccaaaaaacagcaagacacaatacaca

SEQ ID NO: 6 - DNA, 15 bp, Artificial Sequence
  Description of Artificial Sequence Synthetic oligonucleotide
  modified_base (1): a,t,c,g, other or unknown
  modified_base (5)..(6): a,t,c,g, other or unknown
  modified_base (10)..(11): a,t,c,g, other or unknown
  modified_base (15): a,t,c,g, other or unknown
ngaannttcnngaan